(19) United States
(12) Patent Application Publication
(10) Pub. No.: US 2003/0190689 A1
(43) Pub. Date: Oct. 9, 2003

(54) Molecular profiling of disease and therapeutic response using phospho-specific antibodies

(75) Inventors: Katherine Crosby, Middleton, MA (US); Bradley L. Smith, Marblehead, MA (US)

Correspondence Address: James Gregory Cullem, Esq., Intellectual Property Counsel, CELL SIGNALING TECHNOLOGY, INC., 166B Cummings Center, Beverly, MA, 01915, US

(73) Assignee: CELL SIGNALING TECHNOLOGY,INC.

(21) Appl. No.: 10/408,520

(22) Filed: Apr. 7, 2003

Related U.S. Application Data
(60) Provisional application No. 60/370,473, filed on Apr. 5, 2002.

(51) Int. Cl. G01N 33/574

(52) U.S. Cl. 435/7.23

(57) ABSTRACT

The present invention provides methods for identifying the most relevant signal transduction pathway biomarkers of disease progression, outcome, or therapeutic responsiveness, using phospho-specific antibodies in cellular assays to identify proteins whose activity is correlated to the relevant outcome (e.g. therapeutic responsiveness). The invention also provides a method for utilizing correlated biomarker(s) to predict patient response to a therapeutic composition having efficacy against a disease involving altered signal transduction by employing one or more phospho-specific antibodies to detect activation status of such biomarker(s) in cellular assays. Kits for carrying out the methods of the invention are also provided.

### RELATED APPLICATIONS

This application claims priority to U.S. S No. 60/370,473, filed Apr. 5, 2002, now abandoned, the disclosure of which is hereby incorporated by reference herein.

### FIELD OF THE INVENTION

The invention relates generally to signaling proteins and antibodies, and their use to characterize and monitor disease.

### BACKGROUND OF THE INVENTION

The regulation of proteins by secondary modification represents an important cellular mechanism for regulating most aspects of cellular organization and control, including growth, development, homeostasis, and cellular communication. For example, protein phosphorylation plays a critical role in the etiology of many pathological conditions and diseases, including cancer, developmental disorders, autoimmune diseases, and diabetes. In spite of the importance of protein modification, it is not yet well understood at the molecular level. The reasons for this lack of understanding are, first, that the cellular modification system is extraordinarily complex, and second, that the technology necessary to unravel its complexity has not yet been fully developed.

The complexity of protein modification on a proteome-wide scale derives from three factors: the large number of modifying proteins, e.g. kinases, encoded in the genome, the much larger number of sites on substrate proteins that are modified by these enzymes, and the dynamic nature of protein expression during growth, development, disease states, and aging. The human genome encodes, for example, over 520 different protein kinases, making them the most abundant class of enzymes known. See Hunter, Nature 411: 355-65 (2001). Each of these kinases phosphorylates specific serine, threonine, or tyrosine residues located within distinct amino acid sequences, or motifs, contained within different protein substrates. Most kinases phosphorylate many different proteins: it is estimated that one-third of all proteins encoded by the human genome are phosphorylated, and many are phosphorylated at multiple sites by different kinases. See Graves et al., Pharmacol. Ther. 82:111-21 (1999). Many of these phosphorylation sites regulate critical biological processes and may prove to be important diagnostic or therapeutic targets for molecular medicine. For example, of the more than 100 dominant oncogenes identified to date, 46 are protein kinases. See Hunter, supra.

Understanding which proteins, when modified, are relevant to disease will greatly expand our understanding of the molecular mechanisms underlying diseases characterized by signal transduction events. However, at present, the particular modifications and activated signal transduction proteins underlying disease remain largely unknown. Despite this lack of understanding, new therapeutics targeted at a single molecular event or signaling molecule (such as receptor tyrosine kinases) have recently been developed and continue to grow in popularity. The great advantage of targeted therapeutics, which seek to alter the activity of a single protein, over conventional chemotoxic or radiation therapies is that they specifically target the deregulated cell and therefore, should not have the wide cytotoxicity and adverse side effects seen with current therapies. There are currently a large number of targeted drugs in various stages of development with many clinical trials underway. For example, Iressa™, an inhibitor of EGFR, has recently entered clinical trials for the treatment of breast cancer. Similarly, Gleevec®, an inhibitor of BCR/ABL, is now widely used for the treatment of CML.

The successful development, demonstration of efficacy, approval, and use of such targeted drugs will often depend in large part upon the ability of the clinician to determine the activation status of the specific protein that the drug is targeted against. For example, the development of the HER2 inhibitor Herceptin® required the ability to select patients on the basis of their HER2/neu expression (Baselga J. et al. Semin Oncol 1999 26(4 Suppl 12): 78-83). Similarly, the development, approval and use of the targeted BCR/ABL inhibitor Gleevec® has only been possible due to the ability to determine whether a patient's leukemia results from the BCR/ABL translocation (Druker et al., Curr. Oncol. Rep. 3(3): 223-7 (2001)).

In contrast, development of corresponding diagnostic assays to select patients as candidates for targeted therapies, exemplified by the Herceptest™ assay for Herceptin® candidates or the BCR/ABL PCR assay for Gleevec® candidates, has not mirrored the development of the targeted therapeutics. Accordingly, such assays have met with limited success, since they have not necessarily been directed to the most relevant biomarkers of therapeutic response. Assays such as Herceptest™, for example, look only at the expression of the targeted protein and not its activation. However, it is the activity of the protein, and not just its expression, that is actually causing the cellular signaling deregulation and malignancy. As a result, the Herceptest™ diagnostic assay only predicts a successful patient response in approximately 30% of the cases when Herceptin® is used as a single agent (Leyland-Jones. Lancet Oncol (2002) March; 3(3):137-44). This low predictive rate is observed even though all of the patients treated are judged to be over-expressing HER2/neu, demonstrating the significant limitations of this type of diagnostic assay and the need for identifying better biomarkers of responsiveness to therapies like Herceptin®.

Given the complexity of most signaling pathways, downstream pathway markers may prove to be the most predictive of a patient's potential responsiveness to a therapeutic (whether targeted or a general chemotherapeutic), or of disease progression and outcome prognosis. For example, the complexity of HER2/neu and erb-B family signal transduction suggests that downstream pathway markers may be more predictive of patient response to Herceptin® therapy than merely detecting the activity of HER2. Furthermore, since oncogenesis, tumor progression and metastases are thought to require multiple defects in cellular signaling, single-target therapies may not be efficacious in patients having diseases characterized by multiple signaling protein anomalies. Recent results with Gleevec® further indicate that patients will develop resistance to single agent-targeted therapeutics (Sawyers, Science 294(5548): 1834 (2001)). Successful, long-term treatment of such patients will likely require combination therapies targeting multiple signaling pathways and multiple signaling proteins. Identifying the most efficacious therapies for such patients will require a more detailed knowledge of the signaling pathways underlying the patient's disease than is currently available.

Attempts have been made at the genetic level to identify DNA or RNA biomarkers of disease progression. In particular, cDNA gene arrays have been widely used to profile the genetic states of large numbers of genes in various diseases including cancer (Bertucci F. et al., Lab Invest 2003 83(3): 305-16). However, these methods have several shortcomings. Most importantly, gene expression does not necessarily correlate to protein expression, nor does protein expression itself provide an accurate readout of protein activity, in vivo. Indeed, the activity of many proteins, including signal transduction molecules, is modulated by post-translational modification, such as phosphorylation. Therefore, gene arrays are inherently limited in their power to predict cellular response to a therapy or progression of a disease. In addition, current clinical practices rely on techniques that look at small numbers of proteins or genes, not the large number of genes identified by gene arrays. As a result, the list of genes suggested by gene array experiments is typically shortened to a small list that is then verified by in vivo protein studies.

Other approaches have examined the utility of a small number of genes as potential biomarkers for certain diseases. For example, Levine et al. (U.S. Pat. No. 5,843,684) describe a method of diagnosing and predicting prognosis of cancer based upon the expression of p53 and MDM2. Such studies are limited both by their focus on disease prognosis and not response to therapy or relevant targeted therapeutics, and by their focus on protein expression, which may not correlate with protein activity—as has been demonstrated in the case of Herceptin™ response.

Attempts to identify signaling events underlying disease progression or predict therapeutic efficacy at the protein activity level have been made by examining the status of a particular signaling protein utilizing a single modification-specific antibody. For example, assays to monitor the phosphorylation and activation of STAT5 have been described for breast cancer diagnosis and treatment (U.S. Patent Publication No. 20020132274). Another study identified AKT activation in prostate cancer, but did not assess whether such activation was a relevant biomarker of therapeutic responsiveness, or determine the relevance of activation of any other signaling pathway proteins. (Malik et al., Clin. Cancer Res. 8(4):1168-71 (2002)). Alternative methods such as reverse-phase protein arrays and laser micro-dissection have been used to survey multiple proteins in disease (Paweletz et al., Urology 57(4 Suppl 1): 160-3 (2001)). These studies have not examined correlations between signaling molecules and outcome, or attempted to profile the activation of multiple proteins or pathways in disease, and thus have failed to identify the most relevant biomarkers of disease progression or therapeutic response.

All of the foregoing strategies have proven to be of limited utility in identifying the molecular bases of a disease or tumor, determining an effective therapy, or identifying the most relevant biomarkers of disease progression and/or therapeutic responsiveness. It appears that determining the activation status of multiple proteins, both in multiple pathways and at multiple points in the pathways, may, in fact, be required to identify the most useful biomarkers for drug development and testing. Indeed, initial clinical results with targeted therapeutics such as Herceptin® support the conclusion that patient response rates will vary based upon unidentified factors beyond simply the over expression of a single targeted signaling protein (Leyland-Jones, supra.)

Accordingly, new and more powerful techniques are needed for elucidating the molecular bases of disease and identifying the best biomarkers of disease progression and patient response to both targeted therapeutic and chemotherapeutics. In particular, the development of pathway profiling methods at the cellular level, such as immunohistochemistry (IHC), flow cytometry (FC), immunofluorescence (IF), and the like, employing phospho-specific antibodies would be highly desirable. Such cell- or tissue-based methods would enable the rapid analysis of multiple proteins on multiple sequential tissue slices in parallel, as well as a cell-by-cell comparison of protein activation and localization in vivo, and are well suited to high-throughput automation. As new targeted therapeutics continue to be developed and enter clinical trials or use, sets of predictive biomarkers identified by such new techniques would be highly useful in validating specific molecular targets, pre-selecting patients most likely to respond to a specific therapy, and evaluating clinical results based upon knowledge of the most relevant molecular characteristics of the specific disease. Identification of biomarkers predictive of chemotherapeutic response would be highly desirable in order to avoid the prescription of such drugs, and their attendant undesirable effects, to patients that will not respond.

### BRIEF DESCRIPTION OF THE DRAWINGS

FIG. 1. Pathway profiling in LNCaP human prostrate cell line model system.

FIG. 2. Pathway activation in 54 human breast cancer patients. (A) cluster analysis of pathway activation. (B) cluster analysis of patients based upon pathway activation. FIG. 3. Pathway activation in 46 human glioma cancer patients. (A) multi-dimensional plot analysis of pathway activation. (B) cluster analysis of patients based upon pathway activation.

### SUMMARY OF THE INVENTION

The present invention provides, in part, a powerfully informative new method for identifying the most relevant signal transduction pathway biomarkers of disease progression, or therapeutic responsiveness, using panels of phospho-specific antibodies in assays of cellular content, such as IHC, flow cytometry, reversed phase assays, and the like. The invention also provides, in part, a powerful new method for utilizing correlated biomarker(s) to predict patient response to a therapeutic composition having efficacy against a disease involving altered signal transduction by employing one or more phospho-specific antibodies to detect activation status of such biomarker(s) in cellular assays. Kits for carrying out the methods of the invention are also provided.

In accordance with the invention, utilizing a panel of phospho-specific antibodies to profile signal transduction pathway activation in cellular samples from a plurality of patients having a particular disease, coupled with determining correlations among activation statuses of multiple proteins in a pathway and a given outcome (e.g. disease progression, therapeutic responsiveness, survival, etc.) enables the identification of the most relevant and statistically-significant biomarkers of the given outcome. Several novel biomarkers useful for selecting breast cancer patients likely to respond to inhibitors of EGFR or HER2 have now been identified, and are disclosed herein. Identification of the most relevant biomarkers of a given outcome enables previously unavailable methods for accurately predicting or selecting patients likely to respond to a therapy, and for prognosis of disease outcome.

### DETAILED DESCRIPTION OF THE INVENTION

In accordance with the present invention, a powerfully informative new method for profiling signaling pathway activation in order to identify the most relevant biomarkers of disease progression, therapeutic responsiveness, or outcome has now been developed. The method employs a panel of phospho-specific antibodies in a cellular assay (e.g immunofluorescence, IHC, etc.) of a plurality of patient samples in order to identify those signal transduction proteins whose phosphorylation statuses are most highly correlated with an outcome (e.g. therapeutic responsiveness), thus identifying the most relevant biomarkers of the outcome. The invention also provides, in part, a powerful new method for utilizing biomarker(s) correlated to therapeutic response (or disease outcome) to predict patient response to a therapeutic composition (or provide a prognosis of outcome) by using one or more phospho-specific antibodies to detect activation status of such biomarker(s) in a cellular assay. Kits for carrying out these methods are also provided.

It has presently been shown that the in vivo activity of multiple, rather than single, signal transduction proteins must be examined in order to identify the most relevant biomarkers for prognosis of disease outcome or therapeutic responsiveness in diseases, such as cancer, involving altered signal transduction. Furthermore, correlating activation statuses of multiple proteins with a given outcome, such as therapeutic responsiveness or disease progression, is required to determine which signaling events are most relevant to the outcome, and which proteins are therefore the best biomarkers of that outcome.

The use of panels of activation state-specific antibodies in cellular assays of a plurality of patient samples with known outcomes coupled with determining correlations with outcome is a powerfully informative technique having several advantages overcoming the limitations of prior methodologies. The use of panels of antibodies enables the profiling of multiple signaling pathways and multiple signaling proteins in a given disease, thereby identifying multiple signaling events underlying a disease. In contrast, single signaling proteins appear to have limited utility as biomarkers or predictors of an outcome. See Leyland-Jones, supra.; Sawyers, supra.

Cellular analysis, and in particular IHC and flow cytometry, is an accepted clinical procedure (advantageous for clinical/prognostic assays), and enables examination of protein activity at the cell or tissue level (as opposed to protein expression; see Levine, supra.), including the ability to rapidly analyze multiple sequential tissue slices or cells in parallel. In addition, particular cells having activated proteins can be identified, and can, therefore, be directly compared to normal cells to identify differences in in vivo signaling. Further, protein localization within a cell may be determined, in addition to phosphorylation status. Protein localization plays a large role in the regulation of protein function, and may be very important to elucidating molecular bases underlying disease.

Profiling signaling pathway activation in cellular samples from a plurality of patients having a known outcome, such as disease progression, therapeutic response, development of resistance, etc., provides the statistical power necessary to identify relevant signaling events across patient groups, or patient subsets. Correlating multiple protein activation states with a particular outcome ensures that identified biomarkers are, in fact, relevant to that outcome, and thus may be exploited as the best predictive biomarkers. In contrast, the predictive power of individual proteins whose activity is implicated in a disease, but has not been correlated with an outcome or validated as a good biomarker for that outcome, is dubious. See Leyland-Jones, supra.; Sawyers, supra.

Accordingly, in one embodiment, the invention provides a method for identifying protein biomarkers of patient responsiveness (or resistance) to a therapeutic composition having efficacy against a disease involving altered signal transduction, comprising the steps of: (a) obtaining cellular samples from a plurality of patients having the disease, the tissue samples comprising samples from patients (i) treated with the therapeutic composition, (ii) responsive to the therapeutic composition, and (iii) non-responsive (or resistant) to the therapeutic composition; (b) utilizing a panel of phospho-specific antibodies in a cellular assay to detect the phosphorylation statuses of a plurality of signal transduction proteins in the cellular samples; and (c) determining correlations between the phosphorylation statuses of the signal transduction proteins detected in step (b) and responsiveness to the therapeutic composition, wherein one or more significant correlation(s) identifies one or more signal transduction protein(s) as biomarker(s) of patient responsiveness to said therapeutic composition. Certain preferred embodiments of the method are described in more detail below.

As described in more detail in Examples 1 and 2 below, the method described above was employed to identify four novel biomarkers (ERK, estrogen receptor (ER)(Serine118), mTOR, and AKT) useful for predicting breast cancer responsiveness to EGFR or HER2 inhibitors, and four novel biomarkers (PTEN, EGFR, AKT and ERK) useful for predicting glioma cancer.

In another embodiment, the invention provides a method for identifying protein biomarkers useful in disease prognosis, comprising the steps of: (a) obtaining cellular samples from a plurality of patients having a disease involving altered signal transduction, the cellular samples comprising (i) samples from patients having negative and positive disease outcomes, and/or (ii) samples from patients having early-stage and advanced disease; (b) utilizing a panel of phospho-specific antibodies in a cellular assay to detect the phosphorylation statuses of a plurality of signal transduction proteins in the cellular samples; and (c) determining correlations between the phosphorylation statuses of the signal transduction proteins detected in step (b) and progression or outcome of the disease in the patients, wherein one or more significant correlation(s) identifies one or more signal transduction protein(s) as biomarker(s) useful in disease prognosis. Certain preferred embodiments of the method are described in more detail below.

As described in more detail Example 1 below, the method of the invention was employed to identify two novel biomarkers (phosphorylated ER and ERK) useful in prognosis of breast cancer progression and outcome. Phosphorylation of these signaling proteins correlated with breast cancer grade and lymph node status. Tumor grade and lymph node status are important prognostic characteristics of tumors that determine patient outcome.

Biomarkers identified as the most relevant to a given outcome, such as therapeutic response, may be utilized, according to the present invention, to predict or select patients likely to have that outcome. For example, patient response to a therapeutic composition (such as Gleevec®, Iressa®, or a chemotherapeutic) having efficacy against a disease involving altered signal transduction may be predicted utilizing such biomarkers. The exploitation of biomarkers that are correlated to therapeutic responsiveness in a group of patients having a certain disease will enable presently-unavailable levels of predictive accuracy and avoid prescription of therapeutics to patients who will not respond (compare low Herceptest® predictive rates (˜30%) to almost 100% predictive power of exemplary correlated biomarkers identified hereunder and demonstrated in Example 4 (patients found to have a given set of over-expressed or phosphorylated proteins would either relapse or have stable disease or be disease free following therapy, depending on protein expression and phosphorylation).

Accordingly, in one embodiment, the invention provides a method for predicting patient response to a therapeutic composition having efficacy against a disease involving altered signal transduction, comprising the steps of: (a) obtaining at least one cellular sample from a candidate patient having, or a risk of, the disease; (b) utilizing one or more phospho-specific antibodies in a cellular assay to detect the phosphorylation status, in the cellular sample, of one or more signal transduction protein(s) that is/are a correlated biomarker(s) of responsiveness to the therapeutic composition; and (c) determining whether the patient is likely to respond to, or resist, the therapeutic by comparing the phosphorylation status(es) detected in step (b) with a reference biomarker phosphorylation profile characteristic of patients responsive to, or resistant to, the therapeutic composition. Certain preferred embodiments of the method are described in more detail below.

The methods and kits of the present invention enable heretofore unavailable predictive and diagnostic assays pertaining to diseases characterized by signal transduction changes or anomalies, and will be of great value in elucidating the molecular mechanisms of diseases, accelerating drug discovery and approval, and assisting clinicians to prescribe appropriate therapeutics. The further aspects, embodiments, and advantages of the invention are described in more detail below. All references cited herein are hereby incorporated herein by reference.

Definitions

As used throughout this specification, including the claims, the following terms or phrases shall have the meanings indicated:

“therapeutic composition” means any composition of one or more therapeutic compounds, either alone or together (such as in a cocktail of multiple therapeutics); the term encompasses all types of therapeutics, including, but not limited to, small molecule inhibitors, antibody inhibitors, anti-sense or peptide inhibitors, or otherwise, whether “targeted therapeutics” directed to a single protein (such as Iressa™, Gleevec®, and Herceptin®) or agents having more broad activity, such as chemotherapeutic agents;

“disease involving altered signal transduction” means a disease or condition in which altered activity (relative to the non-disease state) of one or more signal transduction proteins is relevant to the genesis and progression of the disease;

“signal transduction protein” means any protein, or peptide fragment thereof, which acts to transmit a signal within a cell (or into a cell) when activated (or deactivated) by post-translational modification (for example, phosphorylation). Signal transduction pathways, or cascades, comprising exemplary signal transduction proteins presently known have been extensively described (See, e.g. Hunter T., Cell 100(1): 113-27 (2000); Cell Signaling Technology, Inc., 2002 Annual Catalog, Pathway Diagrams pgs. 232-253), and include but are not limited to the MAP kinase, AKT, NfkB, WNT, and PKC signaling pathways and their members.

“cellular sample” means any biological sample from an organism containing one or more cell(s), including single cells of any origin, tissue or biopsy samples, or a lysate of any of the foregoing;

“cellular assay” means any assay of cellular protein activity and content, including whole or fixed-cell assays, or cell lysate assays; the terms encompasses, but is not limited to, immunohistochemical (IHC) assays, flow cytometric (FC) assays, immunofluorescent (IF) assays, capture-and-detection assays, and reversed phase assays;

“phospho-specific antibody” means an antibody, whether polyclonal or monoclonal, that binds to a target protein only when phosphorylated at a particular residue or site, and does not substantially bind to the protein when not phosphorylated at that residue or site, or to proteins other than the target protein; the term encompasses humanized antibodies, antibody binding fragments, recombinant antibodies, and the like, as described in more detail in the specification below;

“protein-specific antibody” means an antibody, whether polyclonal or monoclonal, that binds to an unphosphorylated target protein and does not substantially bind to proteins other than the target protein, and is therefore suitable for detecting the presence of the protein in a sample; the term encompasses humanized antibodies, antibody binding fragments, recombinant antibodies, and the like, as described in more detail in the specification below;

“correlated biomarker” means a signal transduction protein whose activity (i.e. phosphorylation state) is significantly correlated with a particular outcome in a group of patients having a disease (for example, therapeutic responsiveness, outcome, prognosis, etc.) and serves, therefore, as a relevant biomarker of the given outcome; the correlation may be either positive or negative and the protein may be activated, or de-activated, by phosphorylation; the term is used interchangeably with “biomarker”;

“responsiveness” as used with respect to a therapeutic composition means either positive responsiveness, non-responsiveness, or resistance;

“panel” with respect to antibodies means two or more antibodies;

“reagent(s) suitable for detecting binding of antibodies” means any material or compound, chemical or biological, suitable for detecting the binding of an antibody to its target; the term encompasses, but is not limited to, fluorescent labels, radio-labels, luminescent reactions, secondary antibodies, and the like; as discussed in more detail in “Cellular Assays” and “Kits” below.

“significant correlation” with respect to a biomarker means a biomarker (or set of biomarkers) the activity of which, when compared to and correlated with an outcome, such as patient response to a therapy or patient prognosis, is statistically different than what would be predicted by chance alone; in the exemplary case of Chi-Squared tests calculations, the statistic characterizes whether the observed distribution of frequencies in a sub-population is significantly different than the overall distribution of frequencies observed in the entire population; the P value that is generally accepted to be statistically relevant is below 0.05, which translates into a confidence level of 95% that the observations are not due to chance alone, and that the correlation is thus significant.

“cluster analysis” means a statistical method to group variables together based upon how they correlate; in the present disclosure, cluster analysis refers to a method to group multiple signal transduction protein biomarkers according to how they are expressed or activated in a particular group of patients, for example, patients having disease mediated through one receptor, as opposed to a second receptor; the cluster analysis may group patients according to the activation or phosphorylation of signaling proteins as well as other molecular biomarkers such as the expression, cellular localization or cleavage of signaling molecules.

Diseases & Pathways

The methods of the invention are applicable to any disease or condition, whether in humans or animals, involving and/or arising, in whole or in part, from altered signal transduction. Cellular signaling pathways are well known in the art (see, e.g., Hunter T., Cell 100(1): 113-27 (2000); Cell Signaling Technology, Inc., 2002 Catalogue, Pathway Diagrams pgs. 232-253). Accordingly, diseases involving or characterized by altered signal transduction may be readily identified, for example, by profiling signal transduction proteins and pathways in diseased tissue with panels of phospho-specific antibodies, as taught herein, and then comparing the pathway activation with normal (non-diseased) tissue pathway activation.

In certain preferred embodiments of the invention, the disease is a cancer, and in a one preferred embodiment, the disease is breast cancer. Other cancers within the scope of the present invention include, but are not limited to, gliomas, lung cancer, colon cancer and prostate cancer. Specific signaling pathway alterations have been described for many cancers, including loss of PTEN and resulting activation of AKT signaling in prostate cancer (Whang Y E. Proc Natl Acad Sci USA Apr. 28, 1998;95(9):5246-50), EGFR overexpression and resulting ERK activation in glioma cancer (Thomas C Y. Int J Cancer Mar. 10, 2003;104(1):19-27) and APC mutation and resulting WNT signaling in colon cancer (Bienz M. Curr Opin Genet Dev 1999 October;9(5):595-603).

Diseases other than cancer involving altered signal transduction are also encompassed by the present invention. For example, it has been shown that diabetes involves underlying signaling changes, namely resistance to insulin and failure to activate downstream signaling through IRS (Burks D J, White M F. Diabetes 2001 February;50 Suppl 1:S140-5). Similarily, cardiovascular disease has been shown to involve hypertrophy of the cardiac cells involving multiple pathways such as the PKC family (Malhotra A. Mol Cell Biochem 2001 September;225(1-):97-107). Inflammatory diseases such as rheumatory arthritis is known to involve the chemokine receptors and disrupted downstream signaling (D'Ambrosio D. J Immunol Methods 2003 February;273(1-2):3-13). The invention is not limited to diseases presently known to involve altered cellular signaling, but includes diseases subsequently shown to involve signaling alterations or anomalies.

The methods (and kits) of the invention may be employed to examine and profile phosphorylation status of any signaling pathway, and any signal transduction protein within such pathways, or collections of such proteins. Single or multiple distinct pathways may be profiled (sequentially or simultaneously), or subsets of proteins within a single pathway or across multiple pathways may be examined (again, sequentially or simultaneously).

Signaling pathways and their protein members have been extensively described. See (Hunter T. Cell Jan. 7, 2000;100(1): 13-27). Exemplary signaling pathways include the following pathways and their protein members: The MAP kinase pathway including ras, raf, MEK, ERK and elk; the AKT pathway including PI-3-kinase, PDK1, AKT and bad; the NfkB pathway including IKKs, IkB and NfkB: the PKC pathway including PI-3-kinase, various PKC isoforms and various PKC substrates such as MARCKS; the WNT pathway including frizzled receptors, beta-catenin, APC and other co-factors and TCF (see Cell Signaling Technology, Inc. 2002 Catolog pages 231-279 and Hunter T., supra.)

Exemplary types of signaling proteins within the scope of the present invention include, but are not limited to, kinases, kinase substrates (i.e. phosphorylated substrates), phosphatases, phosphatase substrates, binding proteins (such as 14-3-3), receptor ligands and receptors (cell surface receptor tyrosine kinases and nuclear receptors)). Kinases and protein binding domains, for example, have been well described (see, e.g., Cell Signaling Technology, Inc., 2002 Catalogue “The Human Protein Kinases” and “Protein Interaction Domains” pgs. 254-279). Although preferred embodiments of the invention assay for, or examine the phosphorylation status of signal transduction proteins, the invention encompasses signal transduction proteins having other post-translationally modifications (e.g. acetylation, glycosylation) identified as relevant to a particular disease (see “Antibodies” section below).

In certain preferred embodiments of the invention, the correlated biomarkers being assayed (or the signaling proteins being examined) are members of the MAP kinase, AKT, NFkB, WNT, and/or PKC signaling pathways. The MAP kinase pathway includes the ras oncogene that is activated in a wide range of cancers (see Cell Signaling Technology, Inc. Catolog, supra. at pages 231-279 and Hunter T, supra. and references therein). The AKT pathway is the central cell survival pathway that is activated by such oncogenic events as overexpression of an upstream receptor tyrosine kinase such as EGFR (ibid) or loss of an upstream regulatory protein such as PTEN (ibid). The NfkB pathway mediates complex cellular response including cell proliferation as well as cell apoptosis all of which are involved in disease (ibid). Activation of the WNT pathway occurs downstream of the APC mutations that are a common cause of colon cancer (ibid). PKC pathway activation is thought to play a role in diseases such as cardiovascular disease and diabetes (ibid). However, the invention is not limited to presently elucidated signaling pathways and signal transduction proteins, and encompasses signaling pathways and proteins subsequently identified.

Therapeutic Compositions

The methods (and kits) of the invention are applicable to any therapeutic composition having efficacy against a disease involving altered signal transduction. Such compositions may include a single therapeutic compound, or multiple therapeutic compounds (such as in a cocktail). Cocktails may include compounds of differing types, for example broad-spectrum chemotherapeutic agents together with targeted small molecule inhibitors. The compositions may include components other than the therapeutic(s), such as stabilizers, buffers, and the like, which formulations are well known in the art and are outside the scope of the present invention.

Therapeutics within the scope of the present invention include, but are not limited to, small molecule inhibitors, antibody inhibitors (including humanized or chimeric antibodies), anti-sense or peptide inhibitors. The therapeutic may be a “targeted therapeutic” directed to a single signaling protein (such as Iressa™, a small molecule inhibitor which targets EGFR), Gleevec®, a small molecule inhibitor which targets BCR-ABL, PDGFR and c-kit), and Herceptin®, a humanized monoclonal antibody which targets HER2). Alternatively, the therapeutic may be a compound having a more broad spectrum of activity, such as chemotherapeutic agents like taxol, cisplatin and methatrexate.

In certain preferred embodiments, the methods of the invention pertain to a therapeutic composition comprising at least one targeted therapeutic. In a preferred embodiment, the targeted therapeutic is a kinase inhibitor. In other preferred embodiments, the methods of the invention pertain to a therapeutic composition comprising at least one chemotherapeutic.

The ability to identify biomarkers of patient responsiveness to targeted therapeutics and/or chemotherapeutic compositions, and to select patients for therapy based on such biomarkers, will be of great value in ensuring that the right patients get the right therapy, and in avoiding unwanted side-effects in patients receiving a therapy to which they will not respond.

Antibodies and Panels

The methods and kits of the invention may employ virtually any phospho-specific antibody capable of detecting a desired signal transduction protein when phosphorylated at a particular residue or site. Phospho-specific antibodies are widely commercially available (e.g. from Cell Signaling Technology, Inc.; BioSource, Inc.; Santa Cruz Biotechnology, Inc.; Upstate Biotechnology, Inc.), and may also be produced by techniques well known in the art (see below).

In the methods and kits for identifying protein biomarkers described herein, panels of phospho-specific antibodies are employed. Such panels may include any collection of two or more phospho-specific antibodies to detect the phosphorylation statuses of two or more target signal transduction proteins. The particular number of antibodies selected for the panel will depend on the signal transduction proteins, pathway or pathways for which profiling is desired. Preferably, phospho-specific antibodies against all known signaling protein members of a given pathway will be employed, however, less than all of the members may be examined. The panel may include phospho-specific antibodies to multiple proteins in two or more distinct pathways. The phosphorylation profile of multiple complete signaling pathways may also be examined. In certain preferred embodiments, the panel comprises two to five phospho-specific antibodies. In other preferred embodiments, the panel comprises five to ten phospho-specific antibodies. In other preferred embodiments, the panel comprises ten to twenty phospho-specific antibodies. In still other preferred embodiments, the panel comprises twenty or more phospho-specific antibodies. The antibodies in a given panel may used sequentially, in tandem, or simultaneously to detect activation statuses of the various targets.

In certain preferred embodiments, the panel of phospho-specific antibodies employed comprises at least one protein that is a member of the MAP kinase, AKT, NFkB, WNT, and/or PKC signaling pathways.

Panels of phospho-specific antibodies used may also include additional non-phospho-specific antibodies or reagents. For example, other modification-specific antibodies may be included, such as acetylation- or nitrosylation-specific antibodies, to detect activation of signal transduction targets having such modifications. Control antibodies may also be included, for example, protein-specific antibodies that detect merely the presence of a given signal transduction protein (not its modification status), or site-specific antibodies that detect a target in its unphosphorylated form.

In the methods and kits for predicting a patient likely to respond to a therapeutic composition described herein, phospho-specific antibodies to one or more signal transduction biomarkers correlated with response to the therapeutic are employed. A single phospho-specific antibody (polyclonal or monoclonal) may be used to detect the phosphorylation status of a single correlated biomarker, for example, if only one such biomarker has been identified as relevant to the disease for which therapy is being considered. Alternatively, two or more (i.e. multliple) phospho-specific antibodies against two or more correlated biomarkers being examined may be employed. The particular number of antibodies selected for predicting patient response in a given case will depend on the number of signal transduction proteins that have been identified as relevant, correlated biomarkers of patient responsiveness to the particular therapeutic composition in a particular disease. One or multiple biomarkers may be identified as relevant predictors of patient response to a particular therapeutic composition for a particular disease. For example, as described in the Examples below, two correlated biomarkers of breast cancer patient responsiveness to EGFR-direct therapeutics were identified, and two correlated biomarkers predict response of such patients to HER2-directed therapeutics.

Phospho-specific antibodies employed to predict patient response may be against a single or multiple correlated biomarker(s) in one pathway (e.g. MAPK pathway) or may be against correlated biomarkers from differing pathways (e.g. MAPK pathway and PKC pathway). Additional phospho-specific antibodies may also be employed (sequentially or simulataneously) to profile the phosphorylation status of additional signal transduction proteins that are not correlated biomarkers, if such additional pathway activation information is desired.

In certain preferred embodiments, a single phospho-specific protein against a single correlated biomarker is employed to predict patient response to a therapeutic composition (having activity against a disease involving altered signal transduction. In another preferred embodiment two or more phospho-specific antibodies against two or more correlated biomarkers are employed. In other preferred embodiments, two to five phospho-specific antibodies against two to five correlated biomarkers are employed. In other preferred embodiments, five to ten phospho-specific antibodies against five to ten correlated biomarkers are employed. Phospho-specific antibodies may be use to detect phosphorylation of correlated biomarkers in the examined cellular sample sequentially, in tandem, or simultaneously to detect activation statuses of the various targets.

In certain preferred embodiments, the phospho-specific antibodies employed comprise at least one target (i.e. the correlated biomarker) that is a member of the MAP kinase, AKT, NFkB, WNT, and/or PKC signaling pathways. However, the methods and kits of the invention for predicting patient response to a therapeutic composition are not limited to presently known signal transduction proteins or pathways, and may be beneficially employed using antibodies to subsequently identified signaling proteins whose phosphorylation is correlated to therapeutic response in a given disease. In other preferred embodiments of the invention, phospho-specific antibodies against ERK, estrogen receptor (ER)(Ser118), mTOR and AKT are employed to select patients likely to respond to EGFR inhibitors (both ERK and ER(Ser118) phosphorylated, but not mTOR and AKT) or HER2 inhibitors (ERK, ER(Ser118), mTOR, and AKT are all activated).

Additional non-phospho-specific antibodies or reagents, or phospho-specific antibodies to targets other than correlated biomarkers, may also be employed in the predictive methods of the invention. For example, other modification-specific antibodies may be included, such as acetylation- or nitrosylation-specific antibodies, to detect activation of signal transduction targets having such modifications. Control antibodies may also be included, for example, protein-specific antibodies that detect merely the presence of a given signal transduction protein (not its modification status), or site-specific antibodies that detect a target in its unphosphorylated form. Additional phospho-specific antibodies may also be employed (sequentially or simulataneously) to profile the phosphorylation status of additional signal transduction proteins that are not correlated biomarkers, if such additional pathway activation information is desired.

Conditions suitable for the binding of antibodies to their signal transduction protein targets are well known in the art, and described in more detail in “Cellular Assays” below.

The methods and kits of the invention are not limited to the use of whole antibodies, but include equivalent molecules, such as protein binding domains or nucleic acid aptamers, which bind, in a phospho-specific manner, to essentially the same phosphorylated epitope to which the particular phospho-specific antibodies bind. See, e.g., Neuberger et al., Nature 312: 604 (1984). Such equivalent non-antibody reagents may be suitably employed in the methods of the invention further described below.

The term “antibody” or “antibodies” refers to all types of immunoglobulins, including IgG, IgM, IgA, IgD, and IgE, including F_{ab} or antigen-recognition fragments thereof. The antibodies may be monoclonal or polyclonal and may be of any species of origin, including (for example) mouse, rat, rabbit, horse, or human, or may be chimeric antibodies. See, e.g., M. Walker et al., Molec. Immunol 26: 403-11 (1989); Morrision et al., Proc. Nat'l. Acad. Sci. 81: 6851 (1984); Neuberger et al., Nature 312: 604 (1984)). The antibodies may be recombinant monoclonal antibodies produced according to the methods disclosed in U.S. Pat. No. 4,474,893 (Reading) or U.S. Pat. No. 4,816,567 (Cabilly et al.) The antibodies may also be chemically constructed by specific antibodies made according to the method disclosed in U.S. Pat. No. 4,676,980 (Segel et al.)

Polyclonal antibodies useful in the practice of the methods and kits of the invention may be produced according to standard techniques by immunizing a suitable animal (e.g., rabbit, goat, etc.) with an antigen encompassing the phosphorylated residue or site to which specificity is desired), collecting immune serum from the animal, separating the polyclonal antibodies from the immune serum, and screening for phospho-epitope specificity in accordance with known procedures. See, e.g., ANTIBODIES: A LABORATORY MANUAL, Chapter 5, p. 75-76, Harlow & Lane Eds., Cold Spring Harbor Laboratory (1988); Czernik, Methods In Enzymology, 201: 264-283 (1991); Merrifield, J. Am. Chem. Soc. 85: 21-49 (1962)).

Monoclonal antibodies suitable for use in the methods and kits of the invention may be produced in a hybridoma cell line according to the well-known technique of Kohler and Milstein. Nature 265: 495-97 (1975); Kohler and Milstein, Eur. J. Immunol. 6: 511 (1976); see also, CURRENT PROTOCOLS IN MOLECULAR BIOLOGY, Ausubel et al. Eds. (1989). Monoclonal antibodies so produced are highly specific, and improve the selectivity and specificity of the therapeutic-response predictive and methods provided by the invention. For example, a solution containing the appropriate antigen (i.e. a desired phospho-epitope of a signal transduction protein) may be injected into a mouse or other species and, after a sufficient time (in keeping with conventional techniques), the animal is sacrificed and spleen cells obtained. The spleen cells are then immortalized by fusing them with myeloma cells, typically in the presence of polyethylene glycol, to produce hybridoma cells. Rabbit fusion hybridomas, for example, may be produced as described in U.S. Pat. No. 5,675,063, C. Knight, Issued Oct. 7, 1997. The hybridoma cells are then grown in a suitable selection media, such as hypoxanthine-aminopterin-thymidine (HAT), and the supernatant screened for monoclonal antibodies having the desired specificity (against the signal transduction protein) by standard techniques. See e.g. Czernik, supra. The secreted antibody may be recovered from tissue culture supernatant by conventional methods such as precipitation, ion exchange or affinity chromatography, or the like.

Monoclonal Fab fragments may also be produced in Escherichia coli by recombinant techniques known to those skilled in the art. See, e.g., W. Huse, Science 246: 1275-81 (1989); Mullinax et al., Proc. Nat'l Acad. Sci. 87: 8095 (1990). If monoclonal antibodies of one isotype are preferred for a particular application, particular isotypes can be prepared directly, by selecting from the initial fusion, or prepared secondarily, from a parental hybridoma secreting a monoclonal antibody of different isotype by using the sib selection technique to isolate class-switch variants (Steplewski, et al., Proc. Nat'l. Acad. Sci., 82: 8653 (1985); Spira et al., J. Immunol. Methods, 74: 307 (1984)).

Cellular Samples & Assay Formats

Cellular samples to be analyzed in the method of the invention may consist of tissue samples taken during the course of surgery, biopsies taken for the sake of patient diagnosis, ductal lavages, fine needle aspirants, blood, serum, urine or other fluid samples or skin, hair follicle or scrapings taken for clinical analysis. Fresh samples may be analyzed by immunohistochemical or immunofluorescent methods on whole cells or by reverse-phase array methods on lysates prepared from the patient samples. Tissue samples may be dispersed, enabling a flow cytometric analysis. Alternatively, the samples may be frozen or fixed using fixation methods well known in the art as described below in the examples. The fixed cells may be paraffin-embedded or used in flow cytometric analyses. The cells derived may also be analyzed as cell smears in which fresh or fixed cells are placed on slides.

Suitable cellular samples from a subject (i.e. biological samples comprising at least one cell or its protein contents) include tissue or tumor samples, individual or multiple cell samples, fine needle aspirate, ductal lavage, bone marrow sample, ascites fluid, urine, lymphatic, or blood samples containing one or more cells, or lysates of the foregoing.

The analysis of the tissue or cell samples may be done by standard immunohistochemical methods well known in the art as described in the examples. This analysis may be done manually or by automatic cell staining instruments. The detection of the bound antibodies may be done with solid substrates or with fluorescent labels. Scoring of the stained tissues or cells may be done manually or by automatic analysis. The fixed cells may be analyzed by flow cytometry using multiple antibodies following standard methods well known in the art.

In certain preferred embodiments of the invention, the cellular sample will be a tumor sample from a cancer patient, for example, a breast cancer patient. In other preferred embodiments, multiple tissue samples are prepared as a tissue microarray for IHC-based staining and analysis. Construction of tissue microarrays is well known in the art (Zhang D. et al. Mod Pathol (2003) January;16(1):79-85).

Phosphorylation status(es) in a cellular sample are examined, in accordance with the methods and kits of the invention, using phospho-specific antibodies in a cellular assay, namely, any assay suitable for detecting in vivo protein activity in a particular cell. Examples of suitable cellular assays include the following preferred assays: immunhistochemistry (IHC), flow cytometry (FC), immunofluorescence (IF) (all of which are whole cell or tissue-based staining assays), and capture-and-detection (e.g. ELISA), or reversed phase assays (which are cell-lysate based assays).

As previously discussed, cellular analysis of protein acitivation has many advantages. Methods like IHC and FC are well-used and accepted clinical procedures, and thus are highly-desirable assay formats for clinical and prognostic assays. Cellular assays enable examination of protein activity at the cell or tissue level (as opposed to genetic or protein expression level; see Levine, supra.), including the ability to rapidly analyze multiple sequential tissue slices or cells in parallel. In addition, particular cells having activated proteins can be identified, and can, therefore, be directly compared to normal cells to identify differences in in vivo signaling. Further, protein localization (which plays a significant role in protein function) within a cell may be determined, in addition to phosphorylation status.

Immunohistochemical (IHC) staining using tissues (either diseased (e.g. a tumor biopsy) or normal) may be carried out according to well known techniques. See, e.g., ANTIBODIES: A LABORATORY MANUAL, Chapter 10, Harlow & Lane Eds., Cold Spring Harbor Laboratory (1988). Briefly, paraffin-embedded tissue (e.g. tumor tissue) is prepared for immunohistochemical staining by deparaffinizing tissue sections with xylene followed by ethanol; hydrating in water then PBS; unmasking antigen by heating slide in sodium citrate buffer; incubating sections in hydrogen peroxide; blocking in blocking solution; incubating slide in primary antibody (i.e. phospho-specific antibodies against signal transduction proteins) and secondary antibody; and finally detecting using ABC avidin/biotin method according to manufacturer's instructions.

Flow cytometry assay may also be employed to determine the activation status of signal transduction proteins and correlated biomarkers. For example, bone marrow cells or peripheral blood cells from patients may be analyzed by flow cytometry for biomarkers of therapeutic response or disease progression, as well as for other markers identifying various hematopoietic cell types. In this manner, activation status of malignant cells may be specifically characterized. Flow cytometry may be carried out according to standard methods. See, e.g. Chow et al., Cytometry (Communications in Clinical Cytometry) 46: 72-78 (2001).

Briefly and by way of example, the following protocol for cytometric analysis may be employed: fixation of the cells with 1% paraformaldehyde for 10 minutes at 37° C. followed by permeabilization in 90% methanol for 30 minutes on ice. Cells may then be stained with the primary phospho-specific antibody or antibodies, washed and labeled with a fluorescent-labeled secondary antibody. Alternatively, the cells may be stained with a fluorescent-labeled primary antibody. The cells would then be analyzed on a flow cytometer (e.g. a Beckman Coulter EPICS-XL) according to the specific protocols of the instrument used.

Immunoassay formats and variations thereof which may be useful for carrying out the methods disclosed herein are well known in the art. See generally E. Maggio, Enzyme-Immunoassay, (1980) (CRC Press, Inc., Boca Raton, Fla.); see also, e.g., U.S. Pat. No. 4,727,022 (Skold et al., “Methods for Modulating Ligand-Receptor Interactions and their Application”); U.S. Pat. No. 4,659,678 (Forrest et al., “Immunoassay of Antigens”); U.S. Pat. No. 4,376,110 (David et al., “Immunometric Assays Using Monoclonal Antibodies”). Conditions suitable for the formation of reagent-antibody complexes are well described. See id. Monoclonal antibodies may be used, for example, in a “two-site” or “sandwich” assay, with a single cell line serving as a source for both the labeled monoclonal antibody and the bound monoclonal antibody. Such assays are described in U.S. Pat. No. 4,376,110. The concentration of detectable reagent should be sufficient such that the binding of phosphorylated target is detectable compared to background.

Alternatively, the biomarkers may be analyzed in an ELISA or reverse-phase array format. For the ELISA format, a capture antibody for each biomarker is affixed to a solid substrate such as a plastic ELISA plate, nitrocellulose membrane or bead. The patient lysate is incubated with the labeled substrate allowing for the capture of the biomarker proteins to the substrate via the capture antibodies. The substrate is then washed. The captured proteins are then detected using a second antibody specific for each protein. The bound detection antibody may be detected by a labeled secondary antibody or by labeling (fluorescent or enzyme) the detection antibody.

In the reverse phase method, lysates of patient samples are fixed to a solid substrate in predetermined locations. The fixed sample is then incubated with the antibodies. After washing, the bound antibodies are detected by various detection methods such as a secondary detection antibodies or by prelabeling the antibodies with fluorescent labels.

Phospho-specific antibodies employed in the methods of the invention may be conjugated to a solid support suitable for a diagnostic assay (e.g., beads, plates, slides or wells formed from materials such as latex or polystyrene) in accordance with known techniques, such as precipitation. Antibodies or equivalent binding reagents, may likewise be conjugated to detectable groups such as radiolabels (e.g., ³⁵S, ¹²⁵I, ¹³¹I), enzyme labels (e.g., horseradish peroxidase, alkaline phosphatase), and fluorescent labels (e.g., fluorescein) in accordance with known techniques.

Alternatively, phospho-specific antibodies employed in cellular assays may be optimized for use in other clinically-suitable applications, for example bead-based multiplex-type assays, such as IGEN, Luminex™ and/or Bioplex™ assay formats, or otherwise optimized for antibody arrays formats.

Calculating Correlations

Any suitable software or algorithm for calculating correlations between the activation (i.e. phosphorylation) of a given signal transduction protein or collection of such proteins may be employed in methods of the invention in identifying relevant biomarkers of disease progression, outcome, prognosis, and/or therapeutic responsiveness.

For example, correlations may be calculated by standard methods, such as the Pearson's tests or Chi-Squared tests. Such methods are well known in the art (see Introduction to Biostatistics, Sokal and Rohlf). The analysis may be done manually or using statistical software such as SYSTAT.

In a preferred embodiment, correlations are determined by performing cluster analysis of protein activity and at least one outcome of interest, such as disease outcome (i.e. survival, death) or therapeutic response or resistance. Cluster analysis uses various statistical methods ranging from simple Pearson correlations to sophisticated mathematical models such as unsupervised learning sets. The outcomes of cluster analysis are often unpredicted by simple analysis of the data. This is especially true of complex data sets such as the data generated by screening hundreds of patient samples on a tissue micro-array using multiple antibodies. Therefore, cluster analysis is a more powerful and informative method than single protein activity correlations that have been previously attempted. As shown in the examples, novel combinations of biomarkers are identified by cluster analysis.

For example, as described in Example 1, cluster analysis of MAP kinase, AKT, STAT, WNT and other pathway activity in breast cancer patients identified phosphorylation of ERK, ER, mTOR and AKT as sufficient to segregate patients into two groups corresponding to EGFR or HER2 expression. Similarily, cluster analysis of AKT, ERK and STAT biomarkers surveyed in Herceptin treated patient samples revealed that the combination of phosphorylated AKT and phosphorylated S6 ribosomal protein predicts patient response and survival to Herceptin combination therapy.

Such correlation analysis enables identification of the best (most highly correlated) biomarkers of disease progression, outcome, or therapeutic responsiveness. Exploitation of such correlated biomarkers in, e.g. predicting therapeutic response of a patient in order to make a treatment determination for that patient, avoids the limitations of present assays based on markers of questionable power (See Herceptest™ discussion above). Identified correlations may be positive or negative: that is, the phosphorylation/activation of a particular biomarker may be negatively correlated with survival, meaning it is associated with non-survival. Similarily, activation or phosphorylation of a particular biomarker may predict a positive patient response or a negative response to a given theratpeutic. The effect of the activation of a given biomarker on a patient response to therapy will depend on the biology of the tumor and the target(s) of the therapy. If the therapy is targeting one oncogene and the patient's tumor is being driven by another oncogene, then it is unlikely that the patient will response well to the therapy. The inverse is true as well.

Preferably, the mostly highly correlated signaling proteins are selected as the best biomarkers of a given outcome. Significant correlations are generally identified as those having a P value of less than 0.05 which means that there is a greater than 95% confidence level that the correlations are not occurring by chance. In the case of the Chi-Squared test, a P value of less than 0.05 indicates that the frequency distribution observed among biomarkers in a subpopulation of patients, for example the patients that response well to a therapy, is significantly different than the frequency distributions observed in the overall population. This conclusion provides the basis for making predictions on how a defined subpopulation of patients will respond to a drug for example. Insignificant correlations indicate proteins not useful as biomarkers as described herein. However, proteins having significant correlations, though not selected as the most highly, and therefore best and most relevant, biomarkers of an outcome (such as survival) may be used, in addition to the best biomarkers, to provide additional information on pathway activation in a given patient.

Correlations may also be determined to identify differences in signaling activity among patient subsets. For example, among breast cancer patients, certain patients will have disease mediated by EGFR signaling, and other patients will have disease mediated by HER2 signaling. Cluster analysis of grouped patient subsets can identify what downstream pathways are activated in subsets of patients. Patients that have tumors driven by AKT pathway activation may response best to drugs such as Rapamycin which targets the mTOR protein which is downstream of AKT. Alternatively, patients that have tumors driven by ERK may benefit most by treatment with ERK pathway inhibitors such as MEK inhibitors. Cluster analysis with multiple biomarkers is required for such an analysis given the complex networks that compose the cellular signaling that drives most tumorigenesis.

Kits

The invention provides, in part, kits for carrying out the methods disclosed herein. In one embodiment, the invention provides a kit predicting patient response to a therapeutic composition having efficacy against a disease involving altered signal transduction, comprising (a) one or more phospho-specific antibodies against one or more signal transduction protein(s) that is/are a correlated biomarker(s) of responsiveness to the therapeutic composition, and (b) one or more additional reagent(s) suitable for detecting binding of the antibodies to the signal transduction protein(s) in a cellular assay. In a preferred embodiment, the therapeutic composition comprises at least one kinase inhibitor or chemotherapeutic. In another preferred embodiment, the kit comprises a plurality of phospho-specific antibodies and protein-specific antibodies against a plurality of correlated biomarkers. Such kits may be used, for example, by a clinician or physician as an aid to selecting an appropriate therapy for a particular patient, for example, a breast cancer patient under consideration for EGFR- or HER2-inhibitor therapy.

As disclosed herein, novel biomarkers of breast cancer responsiveness to EGFR inhibitors and HER2 inhibitors have now been identified. Accordingly, in one preferred embodiment the invention provides a kit for selecting a breast cancer patient likely to respond to a therapeutic composition targeting EGFR or HER2, comprising (a) phospho-specific antibodies against ERK, ER(Ser118), mTOR, and AKT, and (b) one or more additional reagent(s) suitable for detecting binding of these antibodies to their targets in a cellular assay. Activation of both ERK and ER (at Serine 118), but not mTOR and AKT, in a cellular sample from the breast cancer patient identifies the patient as having HER2-mediated cancer and thus likely to respond to a HER2-inhibitor. Activation of ERK, ER(Ser118), mTOR, and AKT in the cellular sample identifies the patient as having EGFR-mediated cancer and thus likely to respond to an EGFR-inhibitor.

In another embodiment, the invention provides a kit for prognosis of disease outcome in a patient having a disease involving altered signal transduction, comprising (a) one or more phospho-specific antibodies against one or more signal transduction protein(s) that is/are a correlated biomarker(s) of outcome or progression of the disease, and (b) one or more additional reagent(s) suitable for detecting binding of said antibodies to the signal transduction protein(s) in a cellular assay. Such kits may used, for example, by a clinician or physician in predicting whether a given patient will survive or present with an aggressive form of a disease, and thus, will aid in determining an appropriately aggressive or passive treatment strategy.

In still another embodiment, the invention provides a kit for identifying protein biomarkers of disease outcome or patient responsiveness to a therapeutic composition having efficacy against a disease involving altered signal transduction, comprising (a) a panel of phospho-specific antibodies against a plurality of signal transduction proteins, and (b) one or more additional reagent(s) suitable for detecting binding of the antibodies to said signal transduction protein(s) in a cellular assay.

In a certain preferred embodiments of these kits, the cellular assay comprises an immunohistochemical (IHC), flow cytometric, immunofluorescent, capture-and-detection, or reversed phase assay, and the kit is optimized for staining or analyzing at least one cellular sample from a patient. In other preferred embodiments, the kit comprises phospho-specific antibodies against one or more members of the MAP kinase, AKT, NFkB, WNT, and/or PKC signaling pathways.

Reagents suitable for detecting binding of the antibodies may, for example, be a second antibody conjugated to a detectable group or label. The kit may include an appropriate assay container, for example, a microtiter plate, slide, etc. The reagents may also include ancillary agents such as buffering agents and protein stabilizing agents, e.g., polysaccharides and the like. The kit may further include, where necessary, other members of the signal-producing system of which system the detectable group is a member (e.g., enzyme substrates), agents for reducing background interference in a test, control reagents, apparatus for conducting a test, and the like. For example, blocking reagents and/or positive and negative controls may be included. Ancillary agents as described above may likewise be included. The test kit may be packaged in any suitable manner, typically with all elements in a single container along with a sheet of printed instructions for carrying out the test. Methods and reagents for carrying out and detecting antibody-protein binding reactions are well known in the art, as described in “Antibodies and Arrays” above.

The following Examples are provided only to further illustrate the invention, and are not intended to limit its scope, except as provided in the claims appended hereto. The present invention encompasses modifications and variations of the methods taught herein which would be obvious to one of ordinary skill in the art.

### EXAMPLE 1

### Identification of Breast and Prostate Cancer Biomarkers Using IHC-Based Analysis

Immunohistochemical (IHC) analysis of paraffin-embedded samples is the most common method for analyzing the pathology of diseased tissues. Determining the molecular pathology of a tumor in order to identify relevant biomarkers of outcome may be accomplished using the methods of the present invention with IHC analysis of paraffin-embedded tissues. IHC analysis of patient tissue samples with phospho-specific antibodies to downstream signaling molecules may be used, for example, to prescreen patients for inclusion in a clinical trial, to follow patients during treatment and to detect resistance to the targeted therapeutic.

The method of the invention was employed using IHC analysis to identify relevant biomarkers of breast cancer outcome and therapeutic response (EGFR and HER2 inhibitors) using tissue microarrays. Tissue micro-arrays are a well-established method to rapidly and uniformly stain large numbers of tissue samples (Zhang et al., Mod. Pathol. 16(1): 79-85 (2003), and may be prepared using commercially available Beecher instruments.

Custom tissue microarrays containing tissue samples from breast cancer patients were obtained commercially (Clinomics, Inc.). The microarrays contained human breast cancer tissues obtained from standard biopsy procedures from patients, subsequently fixed in formallin. The tissue was paraffin-embedded following standard procedures (see ANTIBODIES, A LABORATORY MANUAL, supra.). Alternatively, cultured human LNCaP prostate cancer cells were grown in cell culture and treated with the PI-3-kinase inhibitor LY294002 (LY). The cells were then washed, spun down and the cell pellet was fixed and embedded in paraffin. For IHC staining, 2-4 micron thick slices were cut from the paraffin blocks using a microtome and placed on glass slides. The sections were then de-paraffinized with xylene and ethanol. The tissues were microwaved for 10 minutes in an citrate pH 6.5 buffer for antigen retrieval, or 30 minutes in a pressure cooker for tissue arrays. After a 10 minute incubation in 0.3%H2O2, the sections were blocked in 5% goat serum for 1 hour.

The LNCaP cell slides were then stained with AKT, phospho-AKT, phospho-PKD1, phospho-GSK3, phospho-FKHR, phospho-mTOR, phospho-S6 ribosomal protein and cleaved caspase 3 antibodies (Cell Signaling Technology, Inc.) for 2 hours at room temperature or overnight at 4° C. The breast cancer tissue slides were stained with a variety of receptor tyrosine kinase antibodies and downstream signaling protein phospho-specific antibodies (Cell Signaling Technology, Inc.) as listed in the tables and figures below. After 3 washes in PBS, the slides were then probed with a secondary antibody labeled with biotin. The slides were further developed with a avidin-biotin-HRP reagent (ABC kit) following standard manufacturer procedures. The slides were developed using a HRP substrate, either DAB or NovaRed™ and counterstained with hematoxylin. Positive staining for antibody staining was scored (0-3 or positive-negative) based upon staining intensity, number of cells stained and correct localization of stain. The frequencies of scores were tabulated and the Chi-Squared tests of significance were calculated using standard statistical methods. The cluster analysis was done using well-known publicly available clustering programs such as Cluster and Treeview.

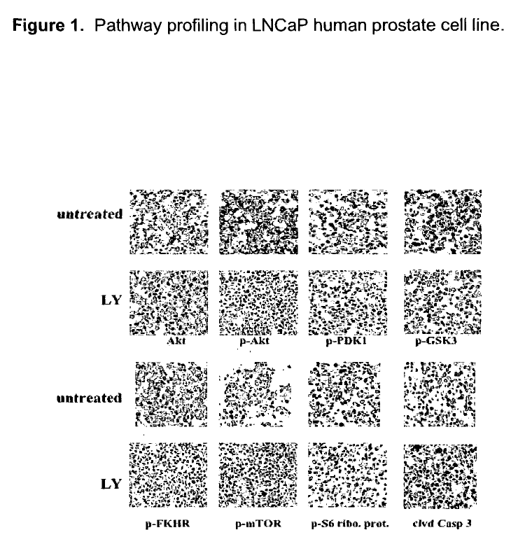

As shown in FIG. 1, AKT pathway activation and it's inhibition can be demonstrated by IHC with phospho-specific antibodies using the PTEN-negative LNCaP cell line. Because these cells are PTEN deficient, the AKT pathway is constitutively activated. The results show that LY inhibition of this pathway is reflected in the loss of staining or phosphorylation of proteins downstream of PI-3-kinase including AKT, PDK1, GSK3, FKHR, mTOR and S6 ribosomal protein. In addition, induction of cleavage of caspase 3 and cellular apoptosis can be observed. These results indicate the usefulness of the method of the invention in profiling pathway activation status, as well as cellular signaling events, in IHC embedded cells or tissues in order to identify relevant biomarkers underlying the disease.

The results of the immunohistochemical study of the breast tumor section arrays were first analyzed for correlations between activation states of proteins and pathological indices including tumor grade and lymph node status (results not shown). Such correlations would indicate the relevance of activation of those proteins with that specific tumor as well as add novel prognostic information. Phosphorylation of ERK and estrogen receptor significantly negatively correlated with tumor grade and lymph node status. Based upon this data, progression of disease may be predicted by monitoring ERK and estrogen receptor phosphorylation. These results further indicate the power of an IHC analysis using panels of phospho-specific antibodies to provide new prognostic information for breast cancer patients.

The immunohistochemical results were then analyzed for statistically significant correlations between activation states of the proteins. Such correlations would indicate that tumors in which one of the proteins is activated would also have the other protein activated. In this way, tumor profiles may be constructed based upon protein activation.

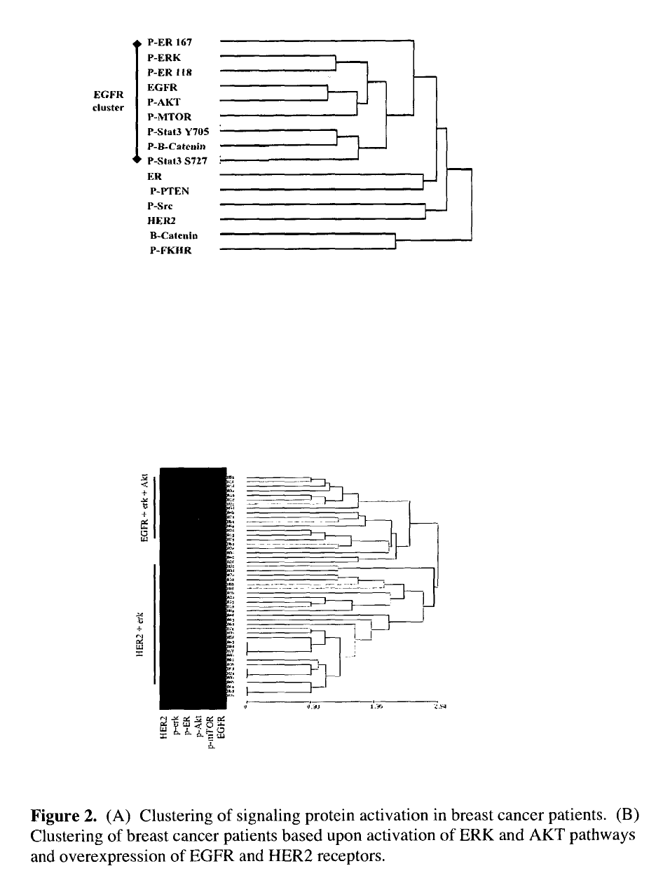

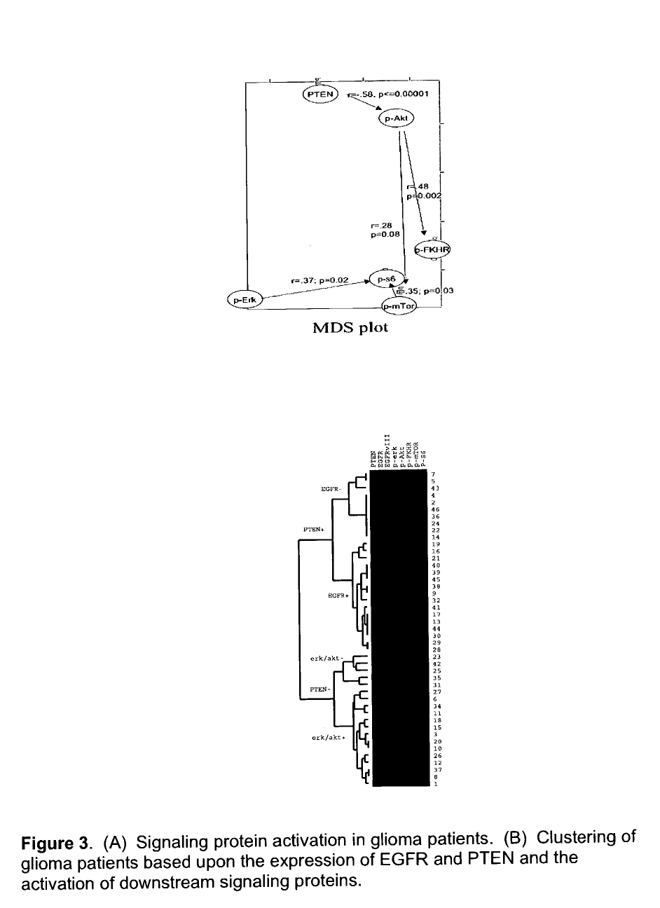

The IHC analysis found that in breast tumors, multiple pathways are typically activated. This observation is not unexpected in that breast tumors often involve the overexpression and activation of receptor tyrosine kinases such as EGFR, which activates multiple pathways including the ERK, AKT STAT3 and beta-catenin pathways. To better understand the results and identify the most useful biomarkers of outcome, a cluster analysis was performed (FIG. 2A). The cluster analysis revealed that EGFR overexpression and downstream signaling may be easily identified by the large EGFR cluster. In this cluster are the proteins whose activation lies downstream of EGFR, including ERK, AKT, STAT3, beta-catenin and other downstream substrates. These results are consistent with what is presently known about EGFR cellular signaling, but further allow for the clustering of patients based upon the IHC results.

For the patient subgroup analysis, a cluster analysis was performed using HER2 and EGFR overexpression and ERK pathway (phospho-ERK and phospho-ER (Ser118)) activation or AKT pathway (phospho-AKT and phospho-mTOR) activation. The results of this cluster analysis clearing segregate patients into two groups; one expresses just EGFR and not HER2 and has both the ERK and AKT pathways activated. The other patient group expresses primarily HER2 and just has the ERK pathway activated. Therefore, the patients may be classified into an EGFR signaling group and a HER2 signaling group based on the identified biomarkers. These biomarkers may now be beneficially employed to directly indicate what breast cancer patients would respond best to an EGFR inhibitor (such as Iressa™) or a HER2 inhibitor (such as Herceptin®).

### EXAMPLE 2

### Identification of Glioma Biomarkers Using IHC-Based Analysis

The power of the profiling methods of the invention were further demonstrated by IHC analysis of a tissue microarray of 46 glioma patients, analyzed using panels of phospho-specific antibodies to identify predictive biomarkers (this work was conducted in a collaborative project with Dr. Charles Sawyers and colleagues at UCLA, and the results are the subject of a pending co-owned provisional patent application (U.S. S No. 60/422,777)). IHC staining analysis was conducted substantially as described in Example 1 above.

The analysis revealed two of the primary oncogenic mechanisms that drive gliomas: EGFR activation or loss of PTEN regulation. The results of the analysis are shown in FIG. 3. FIG. 3A is a multi-dimensional plot, similar based upon a cluster analysis that outlines the relative correlations between the various signaling molecules observed in the patients. As shown, PTEN loss leads to AKT activation and downstream activation of AKT substrates such as FKHR and mTOR. Alternatively, ERK activation (which correlates with EGFR activation; not shown) leads to S6 phosphorylation, which also is regulated by AKT activation. This arrangement of signaling molecules recapitulates what is known of the signaling pathways in cells, therefore validating the IHC analysis.

FIG. 2B presents the corresponding glioma patient cluster analysis based upon the data described in FIG. 2A. Glioma patients may be grouped according to PTEN presence, EGFR expression and ERK and AKT activation. These biomarkers for differing patient groups translate directly into differing treatment regimes: Patients that express PTEN and EGFR and have active downstream signaling would be the best candidates for EGFR inhibitors (such as Iressa™). In contrast, patients that lack PTEN and have active AKT signaling would be most likely to respond to AKT pathway inhibitors (such as rapamycin). These results demonstrate the power of the methods of the invention in identifying biomarkers underlying disease (therefore providing a better understanding of a disease), as well as enabling novel methods to determine the most efficacious therapy for a given patient and/or predict patient response to a given therapeutic composition.

### EXAMPLE 3

### Identification of Biomarkers of HER2 Inhibitor Response in Breast Cancer Patients

Predictive biomarkers of drug response may be identified, according to the methods of the invention, by analyzing tissue samples from patients before therapy and then to compare those results with patient response or survival after therapy. This method was employed with tissue samples from breast cancer patients in order to identify correlated biomarkers predictive of patient response to the HER2-inhibitor, Herceptin®.

The results of the analysis are presented in Tables 1-7. A custom tissue micro-array of 250 breast cancer patients, all treated with a combination of Herceptin (HER2 inhibitor), radiation and chemo-therapies, was commercially obtained (Clinomics, Inc.). The treatment regimes are typical of most therapies prescribed to breast cancer patients at this time. The tissue micro-array was analyzed with panels of antibodies to receptor tyrosine kinases (HER2, EGFR and IGFR), ligands to HER2 and EGFR (NDF, TGF-alpha) and downstream proteins (phosphorylation of AKT, ERK, S6 ribosomal protein, STAT3). The conditions used were as described for Example 1 above.

For the single biomarker analysis, the IHC was scored 0 to 3. For multiple protein analyses the IHC was scored positive or negative for the sake of simplicity. It will be recognized that some statistical power is lost in going to the simpler scoring system, and more accurate (e.g. automated) scoring methodologies may be employed as they are developed in the future. Table 1 presents the results of the analysis of signaling protein phosphorylation compared to patient survival. From the Chi-Squared statistical analysis AKT activation and IGFR expression significantly correlate with patient survival; patients that have AKT activated (score of 1, 2 or 3) have a much lower survival than patients that don't have any AKT phosphorylation. Likewise, patients that overexpress IGFR (score of 3) are far more likely to die than patients whose tumors don't overexpress IGFR.

To further test this method, the data was analyzed using combinations of markers. In this analysis the IHC scores were reduced to positive and negative scores (0 and 1 equals negative and 2 and 3 equals positive). The results of this analysis (Table 2) show that the combination of AKT and S6 ribosomal protein phosphorylation significantly correlate with patient survival. Patients that don't have downstream signaling (AKT and S6 are not phosphorylated) survive the best (40% at the time the clinical data was collected). In comparison, patients that have active AKT and S6 signaling do the worst with only 10% of the patients surviving. Similarily, patient response as defined by disease status also may be predicted using AKT and S6 ribosomal protein phosphorylation (results not shown). Patients that lack AKT and S6 phosphorylation compose 80% of the patients that are disease free after Herceptin® combination therapy. In contrast, patients that have both proteins phosphorylatied compose only 10% of the patient's that are disease free. Accordingly, these biomarkers may be employed to assist in patient prognosis as well as predict patients likely to respond to Herceptin® therapy. Comparing these results with the results obtained with the single biomarker analysis indicates that biomarkers of predictive power may be obtained by considering combinations of multiple biomarkers as opposed to single markers.

### EXAMPLE 4

### Identification of Biomarkers Therapeutic Response in Breast Cancer Patients Segregated by Subgroup

The power of the methods of the invention in identifying relevant biomarkers of a given outcome and predicting outcome based upon the same were further exemplified by an additional analysis of the Herceptin® breast cancer tissue micro-array described in Example 3 above (this work was conducted in a collaborative project with Dr. Sarah Bacus at Ventana Medical Systems, Inc., and the results are the subject of a pending co-owned provisional patent application (U.S. S No. 60/432,942)).

In this analysis, only those breast cancer patients that overexpress HER2 at the 3+ level were included, as these patients are most likely to be given Herceptin® in the clinic. In addition, patient response to the combination Herceptin® therapy was monitored. Patients that had stable disease or were disease free were classified as responders. Patients that showed increases in tumor size were classified as relapsed.

Table 3 presents the results of the comparison of protein phosphorylation and patient response considering the single biomarkers. None of the comparisons were statistically significant. However, when an upstream receptor tyrosine kinase is added to the comparison (Table 4) significant correlations are identified. The combination of EGFR expression and ERK phosphorylation and the combination of IGFR expression and S6 ribosomal protein phosphorylation were identified as statistically significant biomarkers of patient response to therapy. Patients that were EGFR negative but phospho-ERK positive did poorly. Likewise, patients that were IGFR and phospho-S6 positive relapse 92% of the time. Combining three biomarkers gave the most predictive power (Table 5).

When the expression of the upstream ligand, NDF, expression of an upstream receptor tyrosine kinase, IGFR and phosphorylation of a downstream protein, S6 ribosomal protein is considered, patient response may be predicted quite well. For example 100% of the patients that are NDF and phospho-S6 positive and IGFR negative respond. In comparison, 100% of the patients that are NDF negative, phospho-S6 positive or negative and IGFR positive relapse. Similarly, combining NDF and EGFR expression with ERK phosphorylation was capable of predicting 100% patient relapse for significant percentage of the total patient population (28%). Overall, the results with the combinations biomarkers underscore that cancers are often driven by multiple pathways, hence multiple biomarkers must be examined to predict disease outcome or therapeutic response. If a drug is only targeting one of those pathways (such as HER2) than the presence of another pathway that is active (IGFR with an active downstream AKT pathway) will decrease the drug's effectiveness. Identification of such cell signaling information will only be possible with multiple biomarkers capable of determining pathway activation.

The methods of the invention are further exemplified by analysis of samples collected from 7 breast cancer patients before treatment with Herceptin® in combination with chemotherapy (see Table 6). The samples were then analyzed by IHC as described above. Expression of EGFR and IGFR and phosphorylation of AKT, S6 ribosomal protein and ERK were determined. The results indicate that patients who express IGFR and have active downstream signaling (patients #4-7) do not responsd to the therapy. In contrast, patients that either don't express IGFR (patient #2) or express IGFR but don't have active downstream AKT and S6 signaling (patients #1 and #3) do respond. These results are consistent with the tissue micro-array results from the previous examples above. Patient response to a targeted therapy may only be predicted by the use of a combination of molecular biomarkers, detected using phospho-specific antibodies in accordance with the methods disclosed herein.

### List of Tables

Table 1. Analysis of phospho-protein biomarkers and breast cancer patient survival following Herceptin® combination therapy.

Table 2. Analysis of combinations of phospho-protein biomarkers and breast cancer patient survival following Herceptin® combination therapy.

Table 3. Analysis of phosphorylation of a single protein and breast cancer patient response to Herceptin® combination therapy.

Table 4. Analysis of phosphorylation of a single protein and expression of an receptor tyrosine kinase and breast cancer patient response to Herceptin® combination therapy.

Table 5. Analysis of phosphorylation of multiple proteins and breast cancer patient response to Herceptin® combination therapy.

Table 6. Analysis of phosphorylation of downstream signaling proteins, expression of receptor tyrosine kinases and breast cancer patient response to Herceptin® combination therapy.

TABLE 1
Chi-Squared test of significance for protein activation or
expression versus breast cancer patient status (alive or dead)
following herceptin combination therapy.

| | | | | |
|---|---|---|---|---|
| | | | | P value |
| Protein | Score | % alive | % dead | (signif) |

| | | | | | |
|---|---|---|---|---|---|
| p-ERK | 0 | 40 | 60 | 5.3 | 0.15 |
| | 1 | 22 | 78 | | |
| | 2 | 22 | 78 | | |
| | 3 | 24 | 76 | | |
| p-AKT | 0 | 58 | 42 | 41.1 | 0.0001 |
| | 1 | 16 | 84 | | |
| | 2 | 13 | 87 | | |
| | 3 | 15 | 85 | | |
| p-STAT3 | 0 | 29 | 71 | 2.2 | 0.53 |
| | 1 | 32 | 68 | | |
| | 2 | 19 | 81 | | |
| | 3 | 25 | 75 | | |
| p-S6 ribo | 0 | 26 | 74 | 7.1 | (0.07) |
| prot | 1 | 33 | 67 | | |
| | 2 | 17 | 83 | | |
| | 3 | 12 | 88 | | |
| IGFR | 0 | 32 | 68 | 8.4 | 0.04 |
| | 1 | 19 | 81 | | |
| | 2 | 33 | 67 | | |
| | 3 | 9 | 91 | | |

TABLE 2
Chi-Squared test of significance of patient survival versus the
phosphorylation of AKT and S6 ribosomal protein.

| | | | | P value | |
|---|---|---|---|---|---|
| Protein | Score | % alive | % dead | (signif) | |
| p-AKT/p-S6 | neg/neg | 40 | 60 | 18.1 | (0.0001) |
| | neg/pos | 26 | 74 | | |
| | pos/neg | 18 | 82 | | |
| | pos/pos | 10 | 90 | | |

TABLE 3
Downstream protein activation versus patient response following
therapy. Analysis on tissue array samples for which clinical and
Herceptest data was available and who over-expressed HER2/neu.

| | | % | | |
|---|---|---|---|---|
| patient group | n | responders | % relapse | P value |
| p-ERK | 36 | 25% | 75% | 0.43 |
| positive | | | | |
| p-ERK | 39 | 33% | 67% | |
| negative | | | | |
| p-AKT | 24 | 25% | 75% | 0.53 |
| positive | | | | |
| p-AKT | 53 | 32% | 68% | |
| negative | | | | |
| p-S6 positive | 27 | 33% | 67% | 0.74 |
| p-S6 negative | 44 | 30% | 70% | |

TABLE 4
Analysis of receptor and downstream protein activation versus
response in patients following therapy. Analysis on tissue array
samples for which clinical and Herceptest data was available
and who over-expressed HER2/neu.

| | | % | | |
|---|---|---|---|---|
| patient group | n | responders | % relapse | P value |
| EGFR pos/p-ERK pos | 21 | 14% | 86% | 0.04 |
| EGFR pos/p-ERK neg | 19 | 42% | 58% | |
| EGFR neg/p-ERK pos | 9 | 0% | 100% | |
| EGFR neg/p-ERK neg | 14 | 14% | 86% | |
| EGFR pos/p-AKT pos | 17 | 18% | 82% | 0.07 |
| EGFR pos/p-AKT neg | 26 | 38% | 62% | |
| EGFR neg/p-AKT pos | 5 | 20% | 80% | |
| EGFR neg/p-AKT neg | 18 | 6% | 94% | |
| IGFR pos/p-S6 pos | 13 | 8% | 92% | 0.01 |
| IGFR pos/p-S6 neg | 20 | 35% | 65% | |
| IGFR neg/p-S6 pos | 12 | 67% | 33% | |
| IGFR neg/p-S6 neg | 23 | 26% | 74% | |

TABLE 5
Analysis of ligand and receptor expression and downstream
protein activation versus patient response in patients following therapy.
Analysis on tissue array samples for which clinical and Herceptest data
was available and who over-expressed HER2/neu.

| | | % | | |
|---|---|---|---|---|
| patient group | n | responders | % relapse | P value |
| NDF neg/p-S6 pos/IGFR neg | 2 | 50% | 50% | 0.003 |
| NDF neg/p-S6 neg/IGFR neg | 9 | 11% | 89% | |
| NDF neg/p-S6 neg/IGFR pos | 4 | 0% | 100% | |
| NDF neg/p-S6 pos/IGFR pos | 4 | 0% | 100% | |
| NDF pos/p-S6 pos/IGFR neg | 7 | 100% | 0% | |
| NDF pos/p-S6 neg/IGFR pos | 16 | 44% | 56% | |
| NDF pos/p-S6 neg/IGFR neg | 14 | 36% | 64% | |
| NDF neg/p-ERK pos/EGFR neg | 3 | 0% | 100% | 0.08 |
| NDF neg/p-ERK neg/EGFR neg | 4 | 0% | 100% | |
| NDF neg/p-ERK neg/EGFR pos | 10 | 20% | 80% | |
| NDF neg/p-ERK pos/EGFR pos | 6 | 0% | 100% | |
| NDF pos/p-ERK pos/EGFR neg | 5 | 0% | 100% | |
| NDF pos/p-ERK neg/EGFR pos | 13 | 54% | 46% | |
| NDF pos/p-ERK neg/EGFR neg | 6 | 17% | 83% | |
| NDF pos/p-ERK pos/EGFR pos | 18 | 28% | 72% | |

TABLE 6
Receptor tyrosine kinase expression, downstream protein
activation and patient response to therapy in seven breast cancer
patients. Analysis was of whole tissue sections.

| patient | IGFR | EGFR | p-S6 | p-AKT | p-ERK | Response |
|---|---|---|---|---|---|---|
| #1 | + | + | − | − | − | yes |
| #2 | − | + | + | + | + | yes |
| #3 | + | + | − | + | − | yes |
| #4 | + | − | + | + | + | no |
| #5 | + | + | + | + | − | no |
| #6 | + | − | + | + | − | no |
| #7 | + | + | + | + | + | no |

What is claimed is:

1. A method for predicting patient response to a therapeutic composition having efficacy against a disease involving altered signal transduction, said method comprising the steps of:
(a) obtaining at least one cellular sample from a candidate patient having, or at risk of, said disease;
(b) utilizing one or more phospho-specific antibodies in a cellular assay to detect the phosphorylation status, in said cellular sample, of one or more signal transduction protein(s) that is/are a correlated biomarker(s) of responsiveness to said therapeutic composition; and
(c) determining whether said patient is likely to respond to, or resist, said therapeutic by comparing the phosphorylation status(es) detected in step (b) with a reference biomarker phosphorylation profile characteristic of patients responsive to, or resistant to, said therapeutic composition.

2. The method of claim 1, wherein said disease is cancer and wherein said cellular sample is a tumor sample.

3. The method of claim 1, wherein said therapeutic composition comprises at least one targeted therapeutic.

4. The method of claim 3, wherein said targeted therapeutic is a kinase inhibitor.

5. The method of claim 1, wherein said therapeutic composition comprises at least one chemotherapeutic.

6. The method of claim 1, wherein said cellular assay of step (b) comprises an immunohistochemical (IHC), flow cytometric, or immunofluorescent assay.

7. The method of claim 1, wherein said cellular assay of step (b) comprises a capture-and-detection assay or a reversed-phase assay.

8. The method of claim 1, wherein a plurality of phospho-specific antibodies and protein-specific antibodies are employed in step (b) to detect a plurality of correlated biomarkers.

9. The method of claim 1, wherein said correlated biomarker(s) of step (b) comprise at least one protein that is a member of the MAP kinase, AKT, NFkB, WNT, and/or PKC signaling pathways.

10. A kit for predicting patient response to a therapeutic composition having efficacy against a disease involving altered signal transduction, said kit comprising (a) one or more phospho-specific antibodies against one or more signal transduction protein(s) that is/are a correlated biomarker(s) of responsiveness to said therapeutic composition, and (b) one or more additional reagent(s) suitable for detecting binding of said antibodies to said signal transduction protein(s) in a cellular assay.

11. The kit of claim 10, wherein said therapeutic composition comprises at least one kinase inhibitor or chemotherapeutic.

12. The kit of claim 10, wherein said kit comprises a plurality of phospho-specific antibodies and protein-specific antibodies against a plurality of correlated biomarkers.

13. The kit of claim 10, wherein said cellular assay comprises an immunohistochemical (IHC), flow cytometric, or immunofluorescent assay and said kit is optimized for staining of at least one cellular sample from said patient.

14. The kit of claim 10, wherein said cellular assay comprises a capture-and-detection assay or a reversed phase assay and said kit is optimized for analyzing at least one cellular sample from said patient.

15. The kit of claim 10, wherein said correlated biomarker(s) comprise at least one protein that is a member of the MAP kinase, AKT, NFkB, WNT, and/or PKC signaling pathways.

16. A method for identifying protein biomarkers of patient responsiveness to a therapeutic composition having efficacy against a disease involving altered signal transduction, said method comprising the steps of:
(a) obtaining tissue samples from a plurality of patients having said disease, said tissue samples comprising samples from patients (i) treated with said therapeutic composition, (ii) responsive to said therapeutic composition, and (iii) non-responsive to said therapeutic composition;
(b) utilizing a panel of phospho-specific antibodies in a cellular assay to detect the phosphorylation statuses of a plurality of signal transduction proteins in said tissue samples; and
(c) determining correlations between the phosphorylation statuses of said signal transduction proteins detected in step (b) and responsiveness to said therapeutic composition, wherein one or more significant correlation(s) identifies one or more signal transduction protein(s) as biomarker(s) of patient responsiveness to said therapeutic composition.

17. A method for identifying protein biomarkers useful in disease prognosis, said method comprising the steps of:
(a) obtaining tissue samples from a plurality of patients having a disease involving altered signal transduction, said tissue samples comprising (i) samples from patients having negative and positive disease outcomes, and/or (ii) samples from patients having early-stage and advanced disease;
(b) utilizing a panel of phospho-specific antibodies in a cellular assay to detect the phosphorylation statuses of a plurality of signal transduction proteins in said tissue samples; and
(c) determining correlations between the phosphorylation statuses of said signal transduction proteins detected in step (b) and progression or outcome of said disease in said patients, wherein one or more significant correlation(s) identifies one or more signal transduction protein(s) as biomarker(s) useful in disease prognosis.

18. The method of claims 16 or 17, wherein said cellular assay of step (b) comprises an immunohistochemical (IHC), flow cytometric, or immunofluorescent assay.

19. The method of claims 16 or 17, wherein said cellular assay of step (b) comprises a capture-and-detection assay or a reversed phase assay.

20. The method of claims 16 or 17, wherein the determination of correlations in step (c) comprises performing cluster analysis of said phosphorylation statuses.

21. The method of claims 16 or 17, wherein said signal transduction proteins of step (b) comprise proteins that are members of the MAP kinase, AKT, NFkB, WNT, and/or PKC signaling pathways.

22. A kit for prognosis of disease outcome in a patient having a disease involving altered signal transduction, said kit comprising (a) one or more phospho-specific antibodies against one or more signal transduction protein(s) that is/are a correlated biomarker(s) of outcome or progression of said disease, and (b) one or more additional reagent(s) suitable for detecting binding of said antibodies to said signal transduction protein(s) in a cellular assay.

23. A kit for identifying protein biomarkers of disease outcome or patient responsiveness to a therapeutic composition having efficacy against a disease involving altered signal transduction, said kit comprising (a) a panel of phospho-specific antibodies against a plurality of signal transduction proteins, and (b) one or more additional reagent(s) suitable for detecting binding of said antibodies to said signal transduction protein(s) in a cellular assay.

24. The kit of claim 23, wherein said cellular assay comprises an immunohistochemical (IHC), flow cytometric, immunofluorescent, capture-and-detection, or reversed phase assay, and said kit is optimized for staining or analyzing at least one cellular sample from said patient.

25. The kit of claim 23, wherein said signal transduction proteins comprise one or more members of the MAP kinase, AKT, NFkB, WNT, and/or PKC signaling pathways.

26. A method for selecting a breast cancer patient likely to respond to a therapeutic composition targeting Epidermal Growth Factor Receptor (EGFR) or HER2, said method comprising the steps of:
(a) obtaining at least one tumor tissue sample from said patient;
(b) determining, by cellular assay, the phosphorylation statuses of ERK, estrogen receptor (ER)(Ser118), mTOR and AKT in said tissue sample using phospho-specific antibodies; and
(c) comparing the phosphorylation statuses detected in step (b) with a control sample to determine activation of ERK and AKT in said tissue sample relative to said control,
wherein activation of both ERK and ER(Ser118), but not mTOR and AKT, in said tissue sample identifies said patient as having HER2-mediated cancer and thus likely to respond to a HER2-inhibitor, and
wherein activation of ERK, ER(Ser118), mTOR, and AKT in said tissue sample identifies said patient as having EGFR-mediated cancer and thus likely to respond to an EGFR-inhibitor.

27. A kit for selecting a breast cancer patient likely to respond to a therapeutic composition targeting EGFR or HER2, said kit comprising (a) phospho-specific antibodies against ERK, ER(Ser118), mTOR, and AKT, and (b) one or more additional reagent(s) suitable for detecting binding of said antibodies to their targets in a cellular assay.

28. The kit of claim 27, wherein said cellular assay comprises immunohistochemical (IHC), flow cytometric, immunofluorescent, capture-and-detection, or reversed phase staining or analysis of at least one cellular sample from said patient.
